(19) United States
(12) Patent Application Publication
(10) Pub. No.: US 2026/0031233 A1
(43) Pub. Date: Jan. 29, 2026

(54) INTELLIGENT PET BREEDING INTERACTION SYSTEM AND METHOD THEREOF

(71) Applicant: TAMKANG UNIVERSITY, New Taipei City (TW)

(72) Inventor: YI-LUNG LIN, New Taipei City (TW)

(21) Appl. No.: 18/829,357

(22) Filed: Sep. 10, 2024

(30) Foreign Application Priority Data
Jul. 23, 2024 (TW) 113127472

(51) Int. Cl. G16H 50/30

(52) U.S. Cl. CPC G16H 50/30

(57) ABSTRACT

An intelligent pet breeding interaction system includes a storage module, an image capturing module, a processing module, and an artificial intelligence module. The storage module stores a plurality of first postures corresponding to a mental health indicator and a plurality of second postures corresponding to a physical health indicator. The image capturing module monitors a target object to generate a monitoring image. The processing module compares the target object in the monitoring image with the first postures to generate a mental health score and compares the target object in the monitoring images with the second postures to generate a physical health score. The artificial intelligence module receives an inquiry message and generates a response message based on the inquiry message, the mental health score, and the physical health score.

### CROSS REFERENCE TO RELATED APPLICATION

All related applications are incorporated by reference. The present application is based on, and claims priority from, Taiwan Application Serial Number 113127472, filed on Jul. 23, 2024, the disclosure of which is hereby incorporated by reference herein in its entirety.

### TECHNICAL FIELD

The technical field relates to an interaction system, in particular to an intelligent pet breeding interaction system. The technical field further relates to the intelligent pet breeding interaction method of the system.

### BACKGROUND

With the improvement in living standards, many people enjoy keeping pets to enhance their mental health. Pets can provide emotional support to their owners, which can help to alleviate anxiety and depression. However, since animal behavior recognition requires specialized knowledge, most pet owners are unable to effectively identify their pets' behaviors. Currently, there is no effective interaction system that facilitates the interaction between owners and their pets, so most owners are still unable to deeply understand and care for their pets.

Therefore, it has become an important issue to provide an interaction system capable of effectively addressing the aforementioned problems.

### SUMMARY

One embodiment of the disclosure provides an intelligent pet breeding interaction system, which includes a storage module, an image capturing module, a processing module and an artificial intelligence module. The storage module stores a plurality of first postures corresponding to a mental health indicator and a plurality of second postures corresponding to a physical health indicator. The image capturing module monitors a target object to generate a monitoring image. The processing module compares the target object in the monitoring image with the first postures to generate a mental health score and compares the target object in the monitoring images with the second postures to generate a physical health score. The artificial intelligence module receives an inquiry message and generate a response message according to the inquiry message, the mental health score, and the physical health score.

In one embodiment, the mental health indicator comprises an activity level indicator, an interaction behavior indicator, and an emotional indicator. The first postures includes a plurality of activity postures corresponding to the activity level indicator, a plurality of interaction postures corresponding to the interaction behavior indicator, and a plurality of grooming postures corresponding to the emotional indicator. The processing module compares the target object in the monitoring image with the activity postures to generate an activity level score, compares the target object in the monitoring image with the interaction postures to generate an interaction behavior score, and compares the target object in the monitoring image with the grooming postures to generate an emotional score. The processing module generates the mental health score according to the activity level score, the interaction behavior score, and the emotional score.

In one embodiment, the physical health indicator includes a sleep quality indicator, and the second postures include a plurality of sleep postures corresponding to the sleep quality indicator. The processing module compares the target object in the monitoring image with the sleep postures to generate a sleep quality score and generate the physical health score according to the sleep quality score.

In one embodiment, the system further includes a sensing module. The sensing module detects the weight change of an excretion device to generate a daily weight change rate. The storage module stores a daily weight change rate standard value. The processing module compares the daily weight change rate with the daily weight change rate standard value to generate a deviation, and adjust the physical health score according to the deviation

In one embodiment, the artificial intelligence module generates a simulated animation of the target object corresponding to the response message according to the inquiry message, the mental health score, and the physical health score.

Another embodiment of the disclosure provides an intelligent pet breeding interaction method, which includes the following steps: storing a plurality of first postures corresponding to a mental health indicator and a plurality of second postures corresponding to a physical health indicator by a storage module; monitoring a target object to generate a monitoring image by an image capturing module; comparing the target object in the monitoring image with the first postures to generate a mental health score by a processing module; comparing the target object in the monitoring image with the second postures to generate a physical health score by the processing module; and receiving an inquiry message and generating a response message according to the inquiry message, the mental health score, and the physical health score by an artificial intelligence module.

In one embodiment, the mental health indicator comprises an activity level indicator, an interaction behavior indicator, and an emotional indicator. The first postures includes a plurality of activity postures corresponding to the activity level indicator, a plurality of interaction postures corresponding to the interaction behavior indicator, and a plurality of grooming postures corresponding to the emotional indicator. The processing module compares the target object in the monitoring image with the activity postures to generate an activity level score, compares the target object in the monitoring image with the interaction postures to generate an interaction behavior score, and compares the target object in the monitoring image with the grooming postures to generate an emotional score. The processing module generates the mental health score according to the activity level score, the interaction behavior score, and the emotional score.

In one embodiment, the physical health indicator includes a sleep quality indicator, and the second postures include a plurality of sleep postures corresponding to the sleep quality indicator. The processing module compares the target object in the monitoring image with the sleep postures to generate a sleep quality score and generate the physical health score according to the sleep quality score.

In one embodiment, the method further includes the following steps: storing a daily weight change rate standard value by the storage module; detecting a weight change of an excretion device to generate a daily weight change rate by a sensing module; and comparing the daily weight change rate with the daily weight change rate standard value to generate a deviation, and adjusting the physical health score according to the deviation by the processing module.

In one embodiment, the method further includes the following step: generating a simulated animation of the target object corresponding to the response message by the artificial intelligence module according to the inquiry message, the mental health score, and the physical health score.

The intelligent pet breeding interaction system according to the embodiments of the disclosure may have the following advantages:

(1) In one embodiment of the disclosure, the intelligent pet breeding interaction system includes a storage module, an image capturing module, a processing module and an artificial intelligence module. The storage module stores a plurality of first postures corresponding to a mental health indicator and a plurality of second postures corresponding to a physical health indicator. The image capturing module monitors a target object to generate a monitoring image. The processing module compares the target object in the monitoring image with the first postures to generate a mental health score and compares the target object in the monitoring images with the second postures to generate a physical health score. The artificial intelligence module receives an inquiry message and generate a response message according to the inquiry message, the mental health score, and the physical health score. Via this mechanism, the artificial intelligence module can generate the response message that aligns with the target object's mental and physical health status based on the inquiry message, the mental health score, and the physical health score. Therefore, the intelligent pet breeding interaction system allows the user to better understand and care for the target object (pet).

(2) In one embodiment of the disclosure, the artificial intelligence module of the intelligent pet breeding interaction system can also generate a simulated animation of the target object corresponding to the response message based on the inquiry message, the mental health score, and the physical health score. The simulated animation can be generated through augmented reality technology. Thus, the user can not only see the textual content of the response message but also view a simulated animation of the target object speaking and acting. This enhances the interaction by making it more engaging, allowing the user to better understand and care for the target object.

(3) In one embodiment of the disclosure, the mental health score includes an activity level indicator, an interaction behavior indicator, and an emotional indicator. The first postures include a plurality of activity postures corresponding to the activity level indicator, a plurality of interaction postures corresponding to the interaction behavior indicator, and a plurality of grooming postures corresponding to the emotional indicator. The processing module compares the target object in the monitoring image with the activity postures to generate an activity level score, compares the target object in the monitoring image with the interaction postures to generate an interaction behavior score, and compares the target object in the monitoring image with the grooming postures to generate an emotional score. The processing module generates the mental health score based on the activity level score, interaction behavior score, and emotional score. Through this mechanism, the intelligent pet breeding interaction system can generate the mental health score by comprehensively considering multiple different mental health-related indicators in order to more accurately analyze the target object's mental health status.

(4) In one embodiment of the disclosure, the physical health indicator includes a sleep quality indicator. The second postures include a plurality of sleep postures corresponding to the sleep quality indicator. The processing module compares the target object in the monitoring image with the sleep postures to generate a sleep quality score and generates the physical health score based on the sleep quality score. Meanwhile, the intelligent pet breeding interaction system also detects the weight change of an excretion device through a sensing module to generate a daily weight change rate and compares the daily weight change rate with the daily weight change rate standard value to generate a deviation so as to adjust the physical health score based on the deviation. Via this mechanism, the intelligent pet breeding interaction system can generate the physical health score by comprehensively considering multiple different physical health-related indicators with a view to more accurately analyze the target object's physical health status.

(5) In one embodiment of the disclosure, the intelligent pet breeding interaction system integrates artificial intelligence technology with augmented reality technology so as to enable remote interaction between the user and the target object. Accordingly, the intelligent pet breeding interaction system can effectively enhance the emotional connection between the user and the target object.

Further scope of applicability of the present application will become more apparent from the detailed description given hereinafter. However, it should be understood that the detailed description and specific examples, while indicating exemplary embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The disclosure will become more fully understood from the detailed description given herein below and the accompanying drawings which are given by way of illustration only, and thus are not limitative of the disclosure and wherein:

FIG. 1 is the block diagram of an intelligent pet breeding interaction system in accordance with the first embodiment of the disclosure.

FIG. 2 is the block diagram of an intelligent pet breeding interaction system in accordance with the second embodiment of the disclosure.

FIG. 3 is the schematic view of the usage state of an intelligent pet breeding interaction system in accordance with the third embodiment of the disclosure.

FIG. 4 is the flow chart of an intelligent pet breeding interaction method in accordance with the fourth embodiment of the disclosure.

### DETAILED DESCRIPTION

In the following detailed description, for purposes of explanation, numerous specific details are set forth in order to provide a thorough understanding of the disclosed embodiments. It will be apparent, however, that one or more embodiments may be practiced without these specific details. In other instances, well-known structures and devices are schematically shown in order to simplify the drawing.

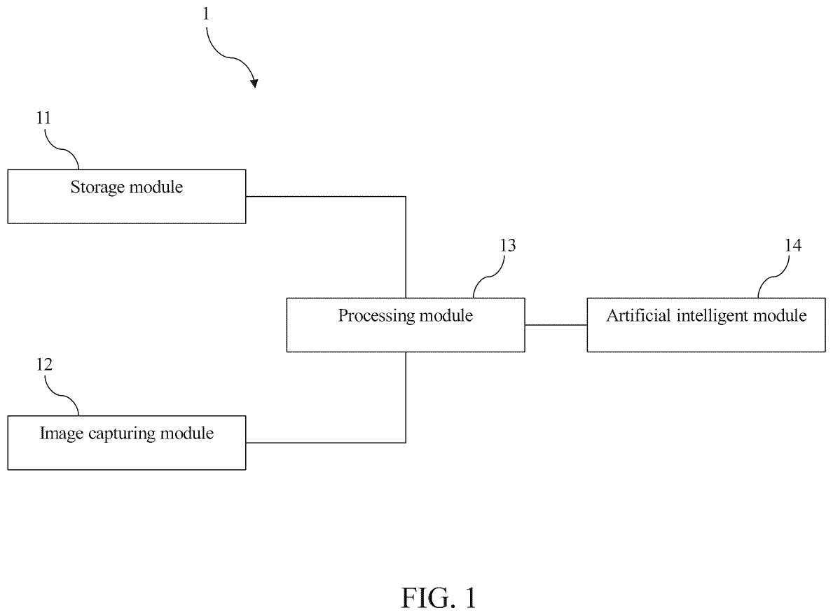

Please refer to FIG. 1, which is the block diagram of an intelligent pet breeding interaction system in accordance with the first embodiment of the disclosure. As shown in FIG. 1, the intelligent pet breeding interaction system 1 includes a storage module 11, an image capturing module 12, a processing module 13, and an artificial intelligence module 14. The storage module 11, image capturing module 12, and artificial intelligence module 14 are connected to the processing module 13. In one embodiment, the storage module 11 may be a flash memory, hard drive, or any currently available storage device. In one embodiment, the image capturing module 12 may be a depth camera, monitor, or other similar device. In one embodiment, the processing module 13 may be a central processing unit (CPU), microcontroller (MCU), application-specific integrated circuit (ASIC), field-programmable gate array (FPGA), or other similar components. In one embodiment, the artificial intelligence module 14 may be a generative AI model, such as LLM, or various currently available machine learning models. In another embodiment, the artificial intelligence module 14 can directly utilize currently available generative AI tools, such as ChatGPT, Bing, etc. The intelligent pet breeding interaction system 1 can be implemented through hardware, software, or a combination of both.

The storage module 11 stores a plurality of first postures corresponding to the mental health indicator and a plurality of second postures corresponding to the physical health indicator. The physical health indicator includes a sleep quality indicator. In this embodiment, the mental health indicator may include an activity level indicator, an interaction behavior indicator, and an emotional indicator. In another embodiment, the mental health indicator may include only a subset of these three indicators. In yet another embodiment, the mental health indicator may also include other indicators related to mental health besides the aforementioned three.

The first postures include postures corresponding to the aforementioned three indicators. In this embodiment, the first postures include a plurality of activity postures corresponding to the activity level indicator, a plurality of interaction postures corresponding to the interaction behavior indicator, and a plurality of grooming postures corresponding to the emotional indicator. These first postures can be obtained through long-term big data analysis to acquire postures corresponding to various behaviors of the target object (a pet, which may include but is not limited to cats, dogs, etc.). The activity level indicator and the activity postures corresponding thereto can reflect the target object's daily activity level (walking, jumping, etc.) or play behaviors (with toys, etc.). The interaction behavior indicator and the interaction postures corresponding thereto can show whether the target object interacts with humans or other animals. The emotional indicator and the grooming postures corresponding thereto can reflect whether the target object frequently grooms itself, which may indicate the target object's emotional state (stress or anxiety).

The image capturing module 12 can continuously monitor the target object to generate a monitoring image.

The processing module 13 compares the target object in the monitoring images with the first postures to generate a mental health score. In this embodiment, the processing module 13 compares the target object in the monitoring images with the activity postures to determine the time and frequency of the target object's postures in the monitoring image matching the activity postures in order to generate an activity level score. The processing module 13 compares the target object in the monitoring images with the interaction postures to determine the time and frequency of the target object's postures in the monitoring image matching the interaction postures so as to generate an interaction behavior score. The processing module 13 compares the target object in the monitoring image with the grooming postures to determine the time and frequency of the target object's postures in the monitoring images matches the grooming postures so as to generate an emotional score. Finally, the processing module 13 generates the mental health score based on the activity level score, interaction behavior score, and emotional score. For example, the processing module 13 can calculate the average of the activity level score, interaction behavior score, and emotional score to obtain the mental health score. For example, the processing module 13 can calculate the mental health score based on the weighted values of the activity level score, interaction behavior score, and emotional score. For example, the processing module 13 can also obtain the weight of each indicator (activity level indicator, interaction behavior indicator, and emotional indicator) for each breed of animal based on breed data (which can be stored in the storage module 11) and perform the weight calculation accordingly. In this way, the processing module 13 can obtain a more accurate mental health score. In another embodiment, after obtaining the weight of each indicator for the corresponding breed, the user can further fine-tune the weight of each indicator according to the pet's individual personality. For example, the user can input fine-tuning values for the weights of the activity level indicator, interaction behavior indicator, and emotional indicator, and the processing module 13 will perform the weight calculation based on the fine-tuned weights.

As previously described, in this embodiment, the physical health indicator includes the sleep quality indicator. In another embodiment, the physical health indicator may also include a body weight indicator, water intake indicator, appetite indicator, etc. The second postures include a plurality of sleep postures corresponding to the sleep quality indicator.

Similarly, the processing module 13 compares the target object in the monitoring image with the second postures to generate a physical health score. In this embodiment, the processing module 13 compares the target object in the monitoring image with the sleep postures to determine the time and frequency of the target object's postures in the monitoring image matching the sleep postures in order to generate a sleep quality score and generate the physical health score based on the sleep quality score. In this embodiment, the processing module 13 uses the sleep quality score as the physical health score. In another embodiment, the processing module 13 may generate the physical health score based on multiple indicators.

Finally, the artificial intelligence module 14 receives the inquiry message and generates a response message and a simulated animation based on the inquiry message, the mental health score, and the physical health score. The simulated animation can be generated through augmented reality technology. For example, the user can inquire about the target object's mood or feelings through an electronic device (such as a smartphone, tablet, etc.), and the electronic device can display the corresponding response message and simulated animation. The response message and simulated animation are generated based on the mental health score and physical health score with a view to fully reflecting the target object's mental and physical health status. In this way, the user can better understand and care for the target object. Additionally, this mechanism also makes the interaction more engaging.

The embodiment just exemplifies the disclosure and is not intended to limit the scope of the disclosure. Any equivalent modification and variation according to the spirit of the disclosure is to be also included within the scope of the following claims and their equivalents.

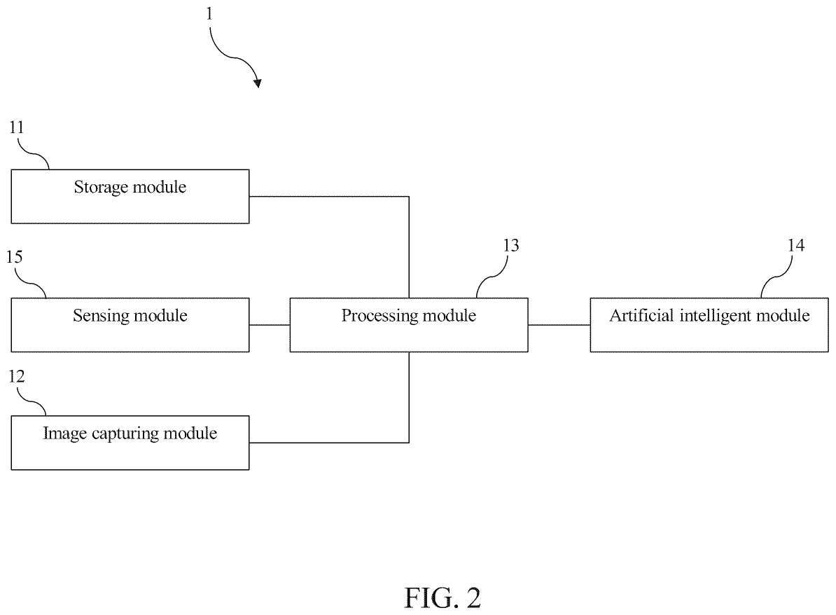

Please refer to FIG. 2, which is the block diagram of an intelligent pet breeding interaction system in accordance with the second embodiment of the disclosure. As shown in FIG. 2, the intelligent pet breeding interaction system 1 includes a storage module 11, an image capturing module 12, a processing module 13, and an artificial intelligence module 14.

The above elements are the same as those in the previous embodiment, so they will not be described herein again. The difference between this embodiment and the previous embodiment is that the intelligent pet breeding interaction system 1 in this embodiment further includes a sensing module 15. The sensing module 15 detects the weight change of an excretion device (such as a litter box) to generate a daily weight change rate. The storage module 11 stores the daily weight change rate standard value. The processing module 13 compares the daily weight change rate with the daily weight change rate standard value to generate a deviation and adjusts the physical health score based on the deviation. This deviation can indicate whether the target object is excreting normally or if there is an abnormal condition. Similarly, the daily weight change rate standard value can be obtained through long-term big data analysis.

Via the above mechanism, the intelligent pet breeding interaction system 1 can generate a physical health score by comprehensively considering multiple different health-related indicators so as to more accurately analyze the target object's physical health status.

The embodiment just exemplifies the disclosure and is not intended to limit the scope of the disclosure. Any equivalent modification and variation according to the spirit of the disclosure is to be also included within the scope of the following claims and their equivalents.

It is worthy to point out that since animal behavior recognition requires specialized knowledge, most pet owners are unable to effectively identify their pets' behaviors. Currently, there is no effective interaction system that facilitates the interaction between owners and their pets, so most owners are still unable to deeply understand and care for their pets. By contrast, according to one embodiment of the disclosure, the intelligent pet breeding interaction system includes a storage module, an image capturing module, a processing module and an artificial intelligence module. The storage module stores a plurality of first postures corresponding to a mental health indicator and a plurality of second postures corresponding to a physical health indicator. The image capturing module monitors a target object to generate a monitoring image. The processing module compares the target object in the monitoring image with the first postures to generate a mental health score and compares the target object in the monitoring images with the second postures to generate a physical health score. The artificial intelligence module receives an inquiry message and generate a response message according to the inquiry message, the mental health score, and the physical health score. Via this mechanism, the artificial intelligence module can generate the response message that aligns with the target object's mental and physical health status based on the inquiry message, the mental health score, and the physical health score. Therefore, the intelligent pet breeding interaction system allows the user to better understand and care for the target object (pet).

Also, according to one embodiment of the disclosure, the artificial intelligence module of the intelligent pet breeding interaction system can also generate a simulated animation of the target object corresponding to the response message based on the inquiry message, the mental health score, and the physical health score. The simulated animation can be generated through augmented reality technology. Thus, the user can not only see the textual content of the response message but also view a simulated animation of the target object speaking and acting. This enhances the interaction by making it more engaging, allowing the user to better understand and care for the target object.

Further, according to one embodiment of the disclosure, the mental health score includes an activity level indicator, an interaction behavior indicator, and an emotional indicator. The first postures include a plurality of activity postures corresponding to the activity level indicator, a plurality of interaction postures corresponding to the interaction behavior indicator, and a plurality of grooming postures corresponding to the emotional indicator. The processing module compares the target object in the monitoring image with the activity postures to generate an activity level score, compares the target object in the monitoring image with the interaction postures to generate an interaction behavior score, and compares the target object in the monitoring image with the grooming postures to generate an emotional score. The processing module generates the mental health score based on the activity level score, interaction behavior score, and emotional score. Through this mechanism, the intelligent pet breeding interaction system can generate the mental health score by comprehensively considering multiple different mental health-related indicators in order to more accurately analyze the target object's mental health status.

Moreover, according to one embodiment of the disclosure, the physical health indicator includes a sleep quality indicator. The second postures include a plurality of sleep postures corresponding to the sleep quality indicator. The processing module compares the target object in the monitoring image with the sleep postures to generate a sleep quality score and generates the physical health score based on the sleep quality score. Meanwhile, the intelligent pet breeding interaction system also detects the weight change of an excretion device through a sensing module to generate a daily weight change rate and compares the daily weight change rate with the daily weight change rate standard value to generate a deviation so as to adjust the physical health score based on the deviation. Via this mechanism, the intelligent pet breeding interaction system can generate the physical health score by comprehensively considering multiple different physical health-related indicators with a view to more accurately analyze the target object's physical health status.

Furthermore, according to one embodiment of the disclosure, the intelligent pet breeding interaction system integrates artificial intelligence technology with augmented reality technology so as to enable remote interaction between the user and the target object. Accordingly, the intelligent pet breeding interaction system can effectively enhance the emotional connection between the user and the target object. As set forth above, the intelligent pet breeding interaction system according to the embodiments of the disclosure can definitely achieve great technical effects.

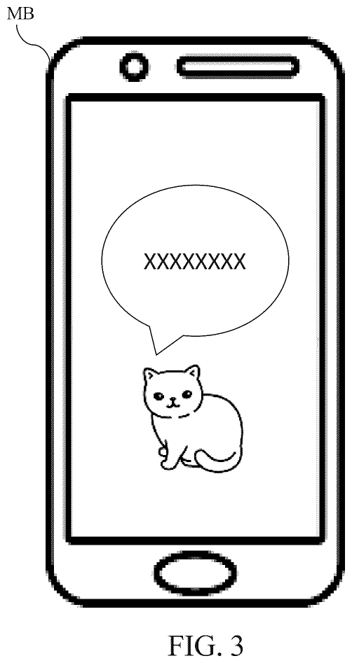

Please refer to FIG. 3, which is the schematic view of the usage state of an intelligent pet breeding interaction system in accordance with the third embodiment of the disclosure. As shown in FIG. 3, the intelligent pet breeding interaction system 1 can be executed or implemented through an electronic device MB, such as a smartphone, tablet, laptop, or similar device.

The user can directly speak a question through the electronic device MB to inquire about the target object's mood or feelings, and the electronic device MB can display a simulated animation and the corresponding response message, which can also be realized in voice form.

Therefore, the intelligent pet breeding interaction system 1 not only allows the user to better understand and care for the target object but also makes the interaction more engaging.

The embodiment just exemplifies the disclosure and is not intended to limit the scope of the disclosure. Any equivalent modification and variation according to the spirit of the disclosure is to be also included within the scope of the following claims and their equivalents.

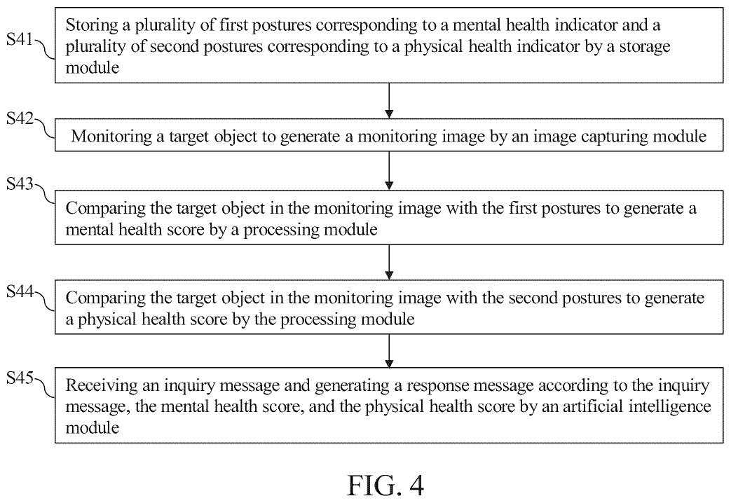

Please refer to FIG. 4, which is the flow chart of an intelligent pet breeding interaction method in accordance with the fourth embodiment of the disclosure. As shown in FIG. 4, the intelligent pet breeding interaction method of this embodiment includes the following steps:

Step S41: storing a plurality of first postures corresponding to a mental health indicator and a plurality of second postures corresponding to a physical health indicator by a storage module.

Step S42: monitoring a target object to generate a monitoring image by an image capturing module.

Step S43: comparing the target object in the monitoring image with the first postures to generate a mental health score by a processing module.

Step S44: comparing the target object in the monitoring image with the second postures to generate a physical health score by the processing module.

Step S45: receiving an inquiry message and generating a response message according to the inquiry message, the mental health score, and the physical health score by an artificial intelligence module.

The embodiment just exemplifies the disclosure and is not intended to limit the scope of the disclosure. Any equivalent modification and variation according to the spirit of the disclosure is to be also included within the scope of the following claims and their equivalents.

Although the operations of the method(s) herein are shown and described in a particular order, the order of the operations of each method may be altered so that certain operations may be performed in an inverse order or so that certain operations may be performed, at least in part, concurrently with other operations. In another embodiment, instructions or sub-operations of distinct operations may be implemented in an intermittent and/or alternating manner.

To sum up, according to one embodiment of the disclosure, the intelligent pet breeding interaction system includes a storage module, an image capturing module, a processing module and an artificial intelligence module. The storage module stores a plurality of first postures corresponding to a mental health indicator and a plurality of second postures corresponding to a physical health indicator. The image capturing module monitors a target object to generate a monitoring image. The processing module compares the target object in the monitoring image with the first postures to generate a mental health score and compares the target object in the monitoring images with the second postures to generate a physical health score. The artificial intelligence module receives an inquiry message and generate a response message according to the inquiry message, the mental health score, and the physical health score. Via this mechanism, the artificial intelligence module can generate the response message that aligns with the target object's mental and physical health status based on the inquiry message, the mental health score, and the physical health score. Therefore, the intelligent pet breeding interaction system allows the user to better understand and care for the target object (pet).

Also, according to one embodiment of the disclosure, the artificial intelligence module of the intelligent pet breeding interaction system can also generate a simulated animation of the target object corresponding to the response message based on the inquiry message, the mental health score, and the physical health score. The simulated animation can be generated through augmented reality technology. Thus, the user can not only see the textual content of the response message but also view a simulated animation of the target object speaking and acting. This enhances the interaction by making it more engaging, allowing the user to better understand and care for the target object.

Further, according to one embodiment of the disclosure, the mental health score includes an activity level indicator, an interaction behavior indicator, and an emotional indicator. The first postures include a plurality of activity postures corresponding to the activity level indicator, a plurality of interaction postures corresponding to the interaction behavior indicator, and a plurality of grooming postures corresponding to the emotional indicator. The processing module compares the target object in the monitoring image with the activity postures to generate an activity level score, compares the target object in the monitoring image with the interaction postures to generate an interaction behavior score, and compares the target object in the monitoring image with the grooming postures to generate an emotional score. The processing module generates the mental health score based on the activity level score, interaction behavior score, and emotional score. Through this mechanism, the intelligent pet breeding interaction system can generate the mental health score by comprehensively considering multiple different mental health-related indicators in order to more accurately analyze the target object's mental health status.

Moreover, according to one embodiment of the disclosure, the physical health indicator includes a sleep quality indicator. The second postures include a plurality of sleep postures corresponding to the sleep quality indicator. The processing module compares the target object in the monitoring image with the sleep postures to generate a sleep quality score and generates the physical health score based on the sleep quality score. Meanwhile, the intelligent pet breeding interaction system also detects the weight change of an excretion device through a sensing module to generate a daily weight change rate and compares the daily weight change rate with the daily weight change rate standard value to generate a deviation so as to adjust the physical health score based on the deviation. Via this mechanism, the intelligent pet breeding interaction system can generate the physical health score by comprehensively considering multiple different physical health-related indicators with a view to more accurately analyze the target object's physical health status.

Furthermore, according to one embodiment of the disclosure, the intelligent pet breeding interaction system integrates artificial intelligence technology with augmented reality technology so as to enable remote interaction between the user and the target object. Accordingly, the intelligent pet breeding interaction system can effectively enhance the emotional connection between the user and the target object.

It will be apparent to those skilled in the art that various modifications and variations can be made to the disclosed embodiments. It is intended that the specification and examples be considered as exemplary only, with a true scope of the disclosure being indicated by the following claims and their equivalents.

What is claimed is:

1. An intelligent pet breeding interaction system, comprising:
a storage module configured to stores a plurality of first postures corresponding to a mental health indicator and a plurality of second postures corresponding to a physical health indicator;
an image capturing module configured to monitor a target object to generate a monitoring image;
a processing module configured to compare the target object in the monitoring image with the first postures to generate a mental health score and compare the target object in the monitoring images with the second postures to generate a physical health score; and
an artificial intelligence module configured to receive an inquiry message and generate a response message according to the inquiry message, the mental health score, and the physical health score.

2. The intelligent pet breeding interaction system as claimed in claim 1, wherein the mental health indicator comprises an activity level indicator, an interaction behavior indicator, and an emotional indicator, and the first postures comprise a plurality of activity postures corresponding to the activity level indicator, a plurality of interaction postures corresponding to the interaction behavior indicator, and a plurality of grooming postures corresponding to the emotional indicator, wherein the processing module is configured to compare the target object in the monitoring image with the activity postures to generate an activity level score, compare the target object in the monitoring image with the interaction postures to generate an interaction behavior score, and compare the target object in the monitoring image with the grooming postures to generate an emotional score, wherein the processing module is configured to generate the mental health score according to the activity level score, the interaction behavior score, and the emotional score.

3. The intelligent pet breeding interaction system as claimed in claim 2, wherein the physical health indicator comprises a sleep quality indicator, and the second postures comprise a plurality of sleep postures corresponding to the sleep quality indicator, wherein the processing module is configured to compare the target object in the monitoring image with the sleep postures to generate a sleep quality score and generate the physical health score according to the sleep quality score.

4. The intelligent pet breeding interaction system as claimed in claim 3, further comprising a sensing module configured to detect a weight change of an excretion device to generate a daily weight change rate, wherein the storage module stores a daily weight change rate standard value, and the processing module is configured to compare the daily weight change rate with the daily weight change rate standard value to generate a deviation, and adjust the physical health score according to the deviation.

5. The intelligent pet breeding interaction system as claimed in claim 1, wherein the artificial intelligence module is further configured to generate a simulated animation of the target object corresponding to the response message according to the inquiry message, the mental health score, and the physical health score.

6. An intelligent pet breeding interaction method, comprising:
storing a plurality of first postures corresponding to a mental health indicator and a plurality of second postures corresponding to a physical health indicator by a storage module;
monitoring a target object to generate a monitoring image by an image capturing module;
comparing the target object in the monitoring image with the first postures to generate a mental health score by a processing module;
comparing the target object in the monitoring image with the second postures to generate a physical health score by the processing module; and
receiving an inquiry message and generating a response message according to the inquiry message, the mental health score, and the physical health score by an artificial intelligence module.

7. The intelligent pet breeding interaction method as claimed in claim 6, wherein the mental health indicator comprises an activity level indicator, an interaction behavior indicator, and an emotional indicator, and the first postures comprise a plurality of activity postures corresponding to the activity level indicator, a plurality of interaction postures corresponding to the interaction behavior indicator, and a plurality of grooming postures corresponding to the emotional indicator, wherein the processing module is configured to compare the target object in the monitoring image with the activity postures to generate an activity level score, compare the target object in the monitoring image with the interaction postures to generate an interaction behavior score, and compare the target object in the monitoring image with the grooming postures to generate an emotional score, wherein the processing module is configured to generate the mental health score according to the activity level score, the interaction behavior score, and the emotional score.

8. The intelligent pet breeding interaction method as claimed in claim 7, wherein the physical health indicator comprises a sleep quality indicator, and the second postures comprise a plurality of sleep postures corresponding to the sleep quality indicator, wherein the processing module is configured to compare the target object in the monitoring image with the sleep postures to generate a sleep quality score and generate the physical health score according to the sleep quality score.

9. The intelligent pet breeding interaction method as claimed in claim 8, further comprising:
storing a daily weight change rate standard value by the storage module;
detecting a weight change of an excretion device to generate a daily weight change rate by a sensing module; and
comparing the daily weight change rate with the daily weight change rate standard value to generate a deviation, and adjusting the physical health score according to the deviation by the processing module.

10. The intelligent pet breeding interaction method as claimed in claim 6, further comprising:
generating a simulated animation of the target object corresponding to the response message by the artificial intelligence module according to the inquiry message, the mental health score, and the physical health score.
